(19) United States
(12) Patent Application Publication
(10) Pub. No.: US 2021/0023127 A1
(43) Pub. Date: Jan. 28, 2021

(54) METHOD TO EXTRACT A PHARMACEUTICAL COMPOSITION FROM A THERAPEUTIC COMPOUND

(71) Applicant: Valentin Matyukhin, Brooklyn, NY (US)

(72) Inventor: Valentin Matyukhin, Brooklyn, NY (US)

(21) Appl. No.: 16/518,055

(22) Filed: Jul. 22, 2019

(51) Int. Cl. A61K 33/04; A61K 31/37; A61K 47/46

(52) U.S. Cl. CPC A61K 33/04; A61K 47/46; A61K 31/37

(57) ABSTRACT

Presented is a method to extract a pharmaceutical composition from a therapeutic compound. The therapeutic compound is a purified shilajit. The composition of shilajit consists of selenium and minerals, humic substances, and dibenzo-alpha-pyrones. In the present embodiment, the therapeutic compound is purified and mixed with at least one plant extract that acts as a natural sweetener to enhance taste of the shilajit, as well as enhance therapeutic efficacy of the pharmaceutical composition obtained from the therapeutic compound.

### FIELD OF THE INVENTION

The present invention relates to a pharmaceutical composition. More particularly, the present invention relates to a pharmaceutical composition containing shilajit or the extract, and a method of extraction of the pharmaceutical composition from a therapeutic compound.

### BACKGROUND

Shilajit is known as a remedy that has a wide spectrum of biological activity on a variety of health-related conditions. The blackish-brown substance obtained from the rocks in high altitudes of the mountains (primarily, Himalayan and Altai) is a product formed of gradual decomposition of certain plants by the action of microorganisms. The composition of shilajit consists of selenium and minerals, humic substances, and dibenzo-alpha-pyrones. The predominant amount of humic substances including fulvic acid and humic acid account for about 60% to 80% of the total nutraceutical compound (raw shilajit compound). Further, studies in the past have revealed that fulvic acid is a humic substance with a smaller molecular weight of about 2 kDa and its soluble properties under different pH conditions provide strong bonds with bioactive molecules and ensure continuous transportation and absorption of shilajit in the small intestine. The Shilajit, (also known as a Mumio) is a natural medicinal food, mainly used to treat people with weakness, inflammation, bone fracture, bleeding and for wound healing. Since there is an insufficient number of medical facilities, about eighty percent of the total population mostly rely on natural products for their primary health care needs. The Shilajit is found to be useful in reducing the time for trauma recovery, bone tissue regeneration, reducing inflammation, improving skin condition, production of testerone, energising and uplifting the organism, stabilizing blood pressure and overall metabolism.

The raw consumption of the Shilajit or unprocessed Shilajit is not preferred for humans as it may contain sand, stones, plant remains and other debris that may make the consumption unsafe. Also, the tasteless shilajit may lead to a reduced intake of the medicine as the taste of the shilajit may not be tolerable by the large portion of people.

In the past, some inventors of various medicinal compositions have envisioned mixing of the processed shilajit with sweeteners or sugar (especially artificial sugar) to enhance the taste of the shilajit (present as one of the ingredients in their composition), in order to increase the consumption of the medicines among the consumers. For example, U.S. Pat. No. 7,250,181 discloses about a polyherbal composition for the treatment of viral infections caused by human immunodeficiency virus (HIV). The patent discloses about polyherbal composition formulation using an extract of purified Shilajit, and plant extracts of Withania somnifera and Mangifera indica. The patent also discloses on adding or mixting sugar or sugar extracts to the polyherbal composition.

WO2018027081 discloses a nutraceutical composition containing a neuroprotecting composition, an anti-inflammatory composition, a cardiovascular protecting composition, an antioxidant composition, and a gelling component in a sufficient amount to provide a cohesive gelled product. This patent application further discloses the nutraceutical composition may include a mitochondria boosting composition comprising acetyl L-carnitine, alpha-lipoic acid, coenzyme Q10 (CoQIO, or ubiquinone), Shilajit extract or powder, nicotinamide riboside (R), vitamin B, vitamin D, omega-3 fatty acids, magnesium, D-ribose, or a derivative or combination thereof. The patent also discloses use of the sweeteners such as sugar, syrup, glucose syrup, corn syrup, high fructose corn syrup, sugar cane syrup, tapioca syrup, juice concentrate, or mixtures thereof with the composition and also use of artificial sweetener.

WO2005041990 discloses about a composition containing Shilajit or the extract thereof, which has the activity of enhancing the metabolic function of the entire body, resulting in an improvement in sexual function and an increase in reproductive function, and thus has effects on nutritional tonic, sexual function improvement, infertility treatment, and the like. This patent application mentions that the liquid formulation of the composition may contain various excipients such as wetting agents, sweeteners, aromatics, and preservatives in addition to generally used simple diluents such as water and liquid paraffin.

Although, the inventors in the past have felt necessity of sweetening the composition by adding some sweeteners in their proposed medicinal compositions, it is found no one did attempt to sweeten the taste of the shilajit itself, specifically during extraction of shilajit in a way not only to preserve medicinal values of the shilajit but to enhance the properties and effectiveness of the shilajit.

Therefore, there is a need to provide a pharmaceutical composition with purified shilajit, which may bring more therapeutic efficacy to the existing medical compositions and encourage the consumption of the medicines among users/consumers.

### BRIEF SUMMARY

Accordingly, proposed is a method to extract a pharmaceutical composition from a therapeutic compound. The therapeutic compound is a purified shilajit. The composition of shilajit consists of selenium and minerals, humic substances, and dibenzo-alpha-pyrones. In the present embodiment, the therapeutic compound is purified and mixed with at least one plant extract that acts as a natural sweetener to enhance taste of the shilajit, as well as enhance therapeutic efficacy of the pharmaceutical composition obtained from the therapeutic compound. In an embodiment, the therapeutic compound and the at least one plant extract corresponds to a similar viscosity of about 10000 cP at the room temperature. In some other embodiment, the therapeutic compound and the at least one plant extract may possess different viscosity at the room temperature. The viscosity depends on the residual moisture content in the therapeutic compound. For example, the plant extract may be added and mixed with any sort of shilajit of different concentration, in such scenario the viscosity of the therapeutic compound and the plant extract will be dissimilar.

Embodiments of the present invention discloses a pharmaceutical composition and method for extraction of the pharmaceutical composition from a therapeutic compound consisting of selenium and minerals, humic substances, and dibenzo-alpha-pyrones.

According to an aspect of the present invention, the pharmaceutical composition comprising a therapeutic compound having selenium and minerals, humic substances, and dibenzo-alpha-pyrones, is characterized in that, the therapeutic compound is mixed with at least one plant extract having polyphenolic composition and other bioactive compounds, such as glyoxal and methylglyoxal, to obtain the pharmaceutical composition for a medical treatment; and wherein the pharmaceutical composition so obtained comprising about 65% of the at least one plant extract, and about 35% of the purified therapeutic compound.

According to this aspect, the therapeutic compound comprises of a purified shilajit. The at least one plant extract is a natural sweetener. The at least one plant extract is selected from a group consisting of a Manuka honey, a Black seed honey, and a combination of the Manuka honey and the Black seed honey added in equal proportions.

According to another aspect of the present invention, the proposed method for extraction of the pharmaceutical composition from the therapeutic compound either make direct use of commercially available purified form of the therapeutic compound or raw form of the therapeutic compound that can be purified using a set of preliminary purification process. The set of preliminary purification process comprises at least crushing or breaking the raw form of the therapeutic compound obtained from rocks in high altitudes of mountains into smaller particles, dissolving the smaller particles of the therapeutic compound in water, and applying multi-stage filtration on the dissolved smaller particles of the therapeutic compound to obtain purified of the therapeutic compounds for further processing.

According to the same aspect, the method further includes adding distilled water to the therapeutic compound (purified form of the therapeutic compound or shilajit), if residual moisture content of the therapeutic compound is found less than 40%.

According the same aspect, the method further includes heating and mixing the therapeutic compound to a temperature ranging between 25° C. to 37° C. using a heating source to form a homogenous composition. The heating source may be a non-electrical heating source, or an electric heating source. According to an example, the non-electrical heating source may be a water bath.

According the same aspect, the method further includes adding and mixing a predefined amount of at least one plant extract to the homogenous composition. The at least one plant extract may be a natural sweetener. The at least one plant extract may be a Manuka honey or a Black seed honey or a combination of a Manuka honey, and a Black seed honey added in equal proportions.

According the same aspect, the method further includes steps of cooling down the homogenous composition with the added at least one plant extract at room temperature of about 70° F., and placing the homogenous composition with the added at least one plant extract into a dehydration system to reduce the residual moisture content of the homogenous composition from 40% or more to 17%-25%. The dehydration system is selected from a group consisting of a vacuum assisted dehydration system, a sunlight assisted dehydration system, and a conventional dehydration system. The homogenous composition is placed into the dehydration system maintaining a temperature lower or equal to 37° C. for about 12-24 hours for reducing the residual moisture content of the homogenous composition from 40% or more to 17% -25%.

According the same aspect, the method further includes steps of remixing the homogenous composition after 12-18 hours to enhance the evenness of texture and prevent precipitation of deposit in the homogenous composition, and storing the homogenous composition at a temperature ranging between 10-2420 C. (50-75° F.) to obtain the pharmaceutical composition. The obtained pharmaceutical composition comprises about 65% of the at least one plant extract, and about 35% of the therapeutic compound.

### BRIEF DESCRIPTION OF THE DRAWING

FIG. 1 illustrates a method to extract a pharmaceutical composition from a therapeutic compound, in accordance with an exemplary embodiment of the present invention; and

### DETAILED DESCRIPTION

An embodiment of this invention, illustrating its features, will now be described in detail. The words “comprising”, “having”, “containing,” and “including,” and other forms thereof, are intended to be equivalent in meaning and be open ended in that an item or items following any one of these words is not meant to be an exhaustive listing of such item or items, or meant to be limited to only the listed item or items. The terms “an” and “a” herein do not denote a limitation of quantity, but rather denote the presence of at least one of the referenced item. The disclosed embodiments are merely exemplary of the invention, which may be embodied in various forms.

Present invention discloses a method to extract a pharmaceutical composition from a therapeutic compound. The therapeutic compound is a purified shilajit. The composition of shilajit consists of selenium and minerals, humic substances, and dibenzo-alpha-pyrones. The predominant amount of humic substances (including fulvic acid and humic acids) account for around 60% to 80% of the total nutraceutical compound. Further, the fulvic acid is a humic substance with a smaller molecular weight of about 2 kDa and its soluble properties under different pH conditions provide strong bonds with bioactive molecules and ensure continuous transportation and absorption of shilajit in the small intestine.

Shilajit, as discussed, is a natural medicinal food, mainly used to treat people with weakness, inflammation, bone fracture, bleeding and for wound healing. Since there is an insufficient number of medical facilities, about eighty percent of the total population mostly rely on the natural products for their primary health care needs. The Shilajit is found to be useful in reducing the time for trauma recovery, bone tissue regeneration, reducing inflammation, improving skin condition, production of testerone, energising and uplifting the organism, stabilizing blood pressure and overall metabolism.

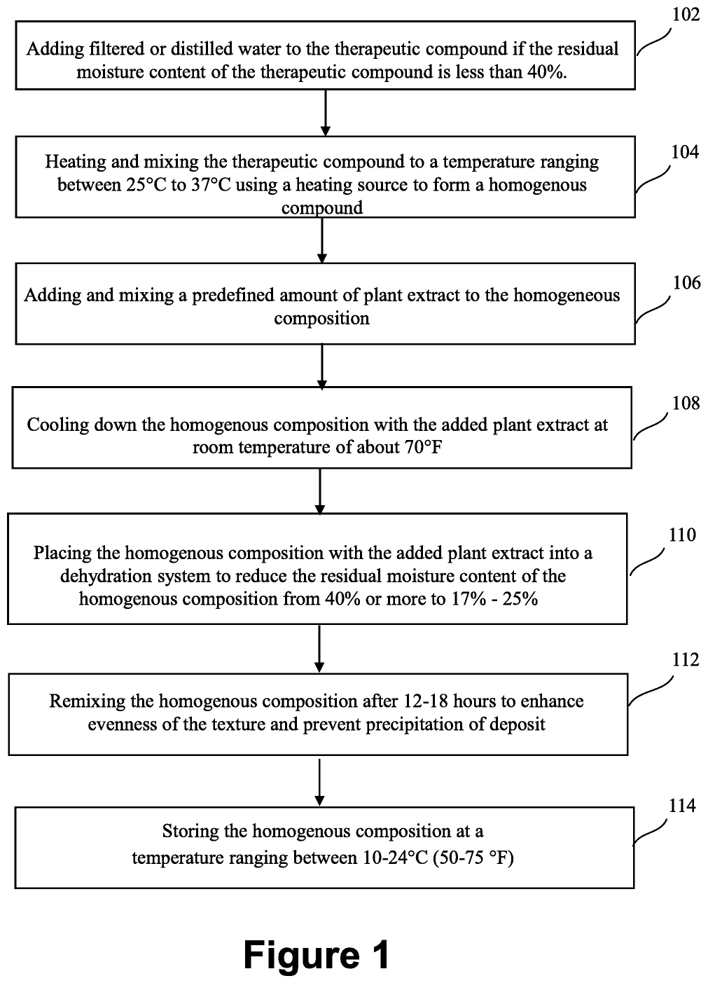

According to the embodiment of the present invention, the therapeutic compound or purified shilajit is mixed with at least one plant extract. The at least one plant extract may be added and mixed with the homogenous composition obtained by heating and mixing the therapeutic compound to a temperature ranging between 25° C. to 37° C. using a heating source that would be discussed in more in FIG. 1. The plant extract is a naturally occurring sweetener that adds sweetness to the pharmaceutical composition. In the present embodiment, the plant extract is a Manuka honey, or a Black seed honey. In one embodiment, either only one type of plant extract (say Manuka honey or Black seed honey) may be added into the purified therapeutic compound or multiple types of plant extracts (say Manuka honey and black seed honey) may be added in equal proportions. However, in either of these two situations, the weight of the plant extracts that is to be added into the purified therapeutic compound should be made equal to the predefined amount of weight determined using the formula:

PE=SE×SC×AC

Where, PE—Total weight of the at least one plant extract (Kg)
SE—Total weight of Shilajit extract/therapeutic compound extract (Kg) extracted using water.
SC—Shilajit concentration (Residual solid content without water content or Total solids content or (1—Residual moisture content)) (weight in %)

### AC—Adjustment Coefficient—7.5

Manuka honey is a monofloral honey derived by European honey bees (Apis mellifera) foraging on the Leptospermum scoparium trees, which are indigenous to New Zealand and Australia. The chemical composition of Manuka Honey, with its non-peroxide antibacterial, antibacterial and antioxidant properties, provide effective treatment of ulcers and other gastrointestinal problems, tissue recovering and wound healing, promotion of oral health, overall rejuvenating and anti-ageing effect on the organism.

The effectiveness of the antimicrobial properties of Manuka honey occurs due to several mechanisms. For example, the inhibition of microbial growth through non-peroxide activities. These non-peroxide activities rely mainly on the action of complex phenols and organic acids, which are referred to as flavonoids. The major flavonoids in Manuka honey are pinobanksin, pinocembrin and chrysin, while luteolin, quercetin, 8-methoxykaempferol, isorhamnetin, kaempferol and galangin have also been identified in minor concentration. Potent bioactive properties of manuka honey are attained to glyoxal, methylglyoxal, and phenolic compounds.

Manuka honey has a high concentration of floral sugars and proteins, enzymes, and amino acids. These sugars are primarily fructose and glucose, with smaller amounts of maltose, sucrose, and isomaltose, and comprise approximately 80% of honey components, with water comprising <18%. Glucose oxidase from the bee crop slowly breaks down glucose into gluconic acid, which lowers the pH of honey, and methylglyoxal, which helps kill bacteria. High sugar and low pH make honey inhibitory to microbial growth.

On the other hand, Black seed honey contains a high percentage of thymoquinone (TQ). N. sativa (Black seed) that has been extensively studied by researchers for its biological activities and therapeutic potential. As seen in the existing studies, the black seed honey has been possess a wide spectrum of activities viz. as diuretic, antihypertensive, antidiabetic, anticancer and immunomodulatory, analgesic, antimicrobial, anthelmintics, analgesics and anti-inflammatory, spasmolytic, bronchodilator, gastroprotective, hepatoprotective, renal protective and antioxidant properties. The polyphenol content of black seed honey, including caffeic acid, chrysin, galangin, kaempferol, apigenin and quercetinadd the antioxidant properties to the black seed honey. Similar attributes of black seed honey and Manuka honey, combined together perform the most effective absorption rate in the human body and boost the immune system and antioxidant levels, which may reduce the cancerous growth. TQ and alpha-hederin, possess remarkable in vitro and in vivo pharmacological activities against a large variety of diseases and found to be relatively safe, and when combined together with Manuka honey and shilajit is expected to increase its performance in the human body for many ailments.

Further, Shilajit can have immune-modulation, antioxidant, diuretic, antihypertensive, and hypoglycemic effects. Shilajit owes the stability of both honey's (Manuka and Black seed honey) active compounds (glyoxal, methylglyoxal, and phenolic compounds) due to their entrapment in the voids (micropores) of the fulvic acids concentrations. A purified fulvic acid carrier is having a sponge-like structure punctured by voids of about 200-1000 A in diameter and an average molecular weight of about 700-2500 to which a water-insoluble and unstable active ingredient added to fill the voids.

Active ingredients of Manuka honey (glyoxal, methylglyoxal, and phenolic compounds) attach to the voids of fulvic acid and arrange the complete module, which then transports and absorbs in the human body. Manuka honey is known to have a relatively low pH (3.5-4.5), which, besides inhibiting microbial growth, stimulates the bactericidal actions of macrophages and, in chronic wounds, reduces protease activity and increases fibroblast activity and oxygenation. Similar properties and effects of shilajit and Manuka honey, bonded together, suggest to improve features of both and can be transmitted and absorbed with higher probability and efficiency. Further in some studies, it is proven that Low to medium molecular weight, hydrophilic, carboxylic-containing molecules and structure of fulvic acid, with its binding ability to attract polyphenolic compounds and methylglyoxal, contained in Manuka honey, not only provide a strong bond but acts as a carrier and transform the antimicrobial compounds for further absorption.

The structure of Fulvic Acid is a mixture of covalently linked phenolic, quinoid, and benzene carboxylic acid compounds. Also, newly formed bonds of compounds of shilajit and Manuka honey provide complementary cooperation and perform increased antibacterial, anti-inflammatory and overall bioactive potent properties on specific health issues, reducing inflammation and microbial growth, effective treatment of ulcer and other gastrointestinal problems, tissue recovering and wound healing, promotion of oral health, and overall rejuvenating and anti-ageing effect on the organism via antioxidants enrichment, delivered into organism.

Traditional medicine and modern research claim Fulvic Acid can modulate the immune system, influence the oxidative state of cells, and improve gastrointestinal function, all of which are hallmarks of diabetes.

Referring to FIG. 1, the method 100 for extracting the pharmaceutical composition by combing the therapeutic compound (purified shilajit) with at least one Manuka honey or Black seed honey or Manuka honey and Black seed honey together is shown. Before the method 100 begins, the invention either makes use of commercially available purified form of the therapeutic compound or proposes a set of preliminary steps using which raw form of the therapeutic compound can be purified before being used in the proposed method 100 to extract a pharmaceutical composition.

The set of preliminary method steps used for purification of the raw purified form of the therapeutic compound or Shilajit includes identifying and collection of the raw therapeutic compound from rocks in high altitudes of mountains. The raw therapeutic compounds obtained are require filtration/purification prior to mixing with the at least one plant extract. During the process of purification, initially the raw form of therapeutic compound is grinded into smaller particles. Next, the grinded particles are dissolved in water for purification. Next, the dissolved grinded particles of the therapeutic compounds undergoes multi-stage filtration process for purifying grinded raw form of therapeutic compounds to obtain purified therapeutic compounds for further processing. Further processing broadly involves, adding and mixing of the at least one plant extract, evaporation of the moisture content from the filtered/purified therapeutic compound mixed with plant extract, and drying up the filtered/purified therapeutic compounds for retail packaging.

Attention is now drawn to FIG. 1, in order to add the at least one plant extract (of Manuka honey and/or black seed honey or both) into the purified therapeutic compound during or prior to evaporation of moisture content from the purified therapeutic compound, the purified therapeutic compound is required to have at least 40% of residual moisture content. In case the residual moisture content of the purified therapeutic compound is less than 40%, the user can add and mix additional filtered or distilled water into the purified therapeutic compound to form a homogenous content that has at least 40% or more residual moisture content (step 102). The purified therapeutic compound is then heated and mixed simultaneously at a temperature ranging between 25 to 37 degrees Celsius using a heating source to form a homogenous compound (step 104). The heating source may be a non-electrical heating source, or an electric heating source. According to an example, the non-electrical heating source may be a water bath. At next step, the at least one plant extract may be added in a predefined amount or ratio into the purified therapeutic compound of the heated mixture (step 106). According to the embodiment, the preferred plant extract is Manuka honey and/or Black seed honey. The predefined amount of the plant extract that can be added into the purified therapeutic compound is determined using the formula:

PE=SE×SC×AC

Where, PE—Total weight of the at least one plant extract (Kg)
SE—Total weight of Shilajit extract/therapeutic compound extract (Kg) extracted using water.
SC—Shilajit concentration (Residual solid content without water content or Total solids content or (1—Residual moisture content)) (weight in %)

### AC—Adjustment Coefficient—7.5

### Example 1

Let's calculate how much (weight) of plant extract may be needed for 50 KG of therapeutic compound that has 30% total solid content concentration.

PE=SE×SC×AC

PE=50×0.3×7.5=112.5 Kgs,

### Example 2

Let's calculate how much (weight) of plant extract may be needed for 2000 KG of shilajit extract prior to the evaporation that has 0.65% total solid content concentration.

PE=SE×SC×AC

PE=1000×0.0065×7.5=48.75 Kgs.

Further, according to the embodiment, either only one type of plant extract (say Manuka honey or Black seed honey) may be added into the purified therapeutic compound or multiple types of plant extracts (say Manuka honey and Black seed honey both) may be added in equal proportions. However, in either of these two situations, the weight of the plant extracts that is added into the purified therapeutic compound should be made equal to the predefined amount of weight determined using the formula: PE=SE×SC×AC. Considering above examples (Example 1 and Example 2):

### Example 1

Either 112.5 kgs of Manuka honey or black seed honey may be added or 56.25 kgs of each of these two honeys may be added.

### Example 2

Either 48.75 kgs of Manuka honey or black seed honey may be added or 24.375 kgs of each of these two honeys may be added.

Next, upon adding the predetermined amount of the at least one plant extract with the purified therapeutic compound during the process of evaporation of the moisture content (the step 106), the homogenous composition with the added plant extract is left at a room temperature of about 70° F. to cool down (step 108).

Next, at step 110, the homogenous composition obtained from the combination of the purified therapeutic compound and the plant extract preferably the Manuka honey is placed into a dehydration system for certain time say 12-24 hours to reduce the moisture content. The dehydration system may include a vacuum assisted dehydration system, a sunlight assisted dehydration system, or a conventional dehydration system. In an example, the dehydration system may be a freeze dry system. The homogenous composition is placed into the dehydration system maintaining a temperature lower or equal to 37° C. for about 12-24 hours for reducing the residual moisture content of the homogenous composition from 40% or more to 17%-25%.This reduction of the moisture content is important to prevent fermentation of the Manuka honey that may occur at higher moisture content. This step creates stable molecular bonds, as well as form the composition's texture and taste due to the natural crystallization process of the Manuka honey.

Later in the next step, the homogenous composition is remixed after 12-18 hours to enhance the evenness of the texture and prevent precipitation of deposit in the homogenous composition (step 112).

Further, in the next step, the homogenous composition is stored at a temperature ranging between 10-24° C. (50-75° F.) to obtain the pharmaceutical composition (step 114). In the present embodiment, the pharmaceutical composition is stored at the maintained air temperature of 10-24° C. (50-75° F.) in airtight glass jars to prevent oxidation.

The obtained pharmaceutical composition following the method described in FIG. 1 consists of about 65% of the plant extract, and rest about 35% of purified shilajit.

The combined Manuka honey and the shilajit form strong antioxidant capacities, preventing cell damage. The beneficial effect of honey on human health derives mainly from its content in phenolic compounds. These substances have been recognized as the main responsible for the antioxidant activity of honey that is primarily associated with the ability of free radical scavengers, through the formation of more stable and less toxic molecules. Phenolic compounds stabilize free radicals when they give off hydrogen from one of their hydroxyl group; the degree of activity is related to the number of their hydroxyl groups. The AOC (antioxidant capacity) of honey is given primarily by phenolic compounds, enzymes, amino acids, and carotenoids also contribute to this ability. Radical scavenging and protection against the lipid peroxidation of honey can reduce and prevent diseases and physiological situations where oxidative stress plays an important role.

The consumption of shilajit positively enhances antioxidant activity, immunity, and disease resistance. According to some studies, the antioxidant property of processed shilajit was compared to unprocessed shilajit and vitamin C (ascorbic acid). Processed shilajit exhibited the significant antioxidant activity of itself and also could regenerate (recycle) ascorbic acid after it had neutralized free radicals. The dihydroxybenzoic-alpha-pyrones in shilajit caused recycling (regeneration) of ascorbic acid.

The shilajit is tested for its ability to neutralize sulphite anion, hydroxyl and nitric oxide free radicals. The chemical polymerization by free radicals is measured with and without processed shilajit. Further in some studies, it is proven that processed shilajit provides almost complete protection of methyl methacrylate against hydroxyl radical-induced polymerization and significantly inhibited the polymerization of methyl methacrylate by the sulphite free radical. Processed shilajit efficiently traps nitric oxide free radicals, the concentration-dependent antioxidant effects. Higher concentrations of the processed shilajit provides greater free radical protection.

The components of the invention tend to perform the anti-ulcer activity. The shilajit increases the thickness of the protective layer of mucus secreted by the mucus-secreting cells in the lining of the stomach. This protects the wall of the stomach from the acid preventing and allowing ulcers to heal, and allows proper digestion and assimilation of food. The Manuka honey has been shown to specifically decrease the inflammatory response associated with ulcerative colitis, an inflammatory intestine disease characterized by an overexpression of inflammatory cells, in embryonic kidney cell lines. The anti-inflammatory effect by the Manuka honey was strongest in the presence of the Pam3CSK4 ligand, indicating that the honey act through the TLR1/TLR2 signalling pathway. The anti-inflammatory activity of Manuka honey is, therefore, pathway specific.

The Manuka honey and the Shilajit is known as an antimicrobial and an anti-inflammatory remedy since ancient times. The antibacterial properties of honey and shilajit demonstrate the minimum inhibitory concentration (MIC), and the minimum bactericidal concentration (MBC) of substances with diverse bacterial agents. This ability is mainly due to some physical properties of this matrix, such as low water activity (Aw), high osmotic pressure, low pH, and moderate protein and mineral content, which prevent bacterial growth.

Further, in addition to these physical properties, the antimicrobial activity of honey is also due to the glucose oxidase, H2O2, and to some phenolic compounds such as pinocembrin, syringic acid, and some others compounds and role of methylglyoxal in Manuka honey (Leptospermum scoparium), since it is believed to be the most responsible for the non-peroxide antibacterial activity of honey. Low pH of the shilajit and the Manuka honey and ability of the Fulvic Acid to attract bioactive compounds, bond and transport them increase the effectiveness of the invention. The activity of the method linked to enhancing antibacterial capacity and successful reduction of inflammation crater, healing wounds, gastrointestinal issues, ulcers and prompt oral health.

Likewise, combined Black seed honey and the Shilajit, or combined equal proportions of Manuka honey and Black seed honey with the Shilajit also produces enhanced therapeutic benefits among consumers. Adding honey to the shilajit not only enhances taste, but also provides additional benefit of keeping the extracted composition/purified shilajit consistent (non-dry) over a longer period of time when compared to regular shilajit purified or extracted using just water.

By way of non-limiting example, the pharmaceutical composition can be consumed in many forms such as supplements and in different therapeutically amount/doses. For example, the pharmaceutical composition can be provided as a syrup or a paste form. It may be obvious to a person skilled in the art to provide the pharmaceutical composition in many other forms including pill or tablet, powder etc., without departing from the scope of this disclosure.

Further, in a non-limiting example, certain cases showcasing the advantages and effects of the consumption of the pharmaceutical composition in various group of age limits are illustrated below.

Case 1: A 35 year old female, with severe bruises and hickeys on her face. After consuming the supplement for a week reported significant decrease of the bruises and hickeys and overall improvements of the skin tissues.

Case 2: A 30 years old male soccer player had an ankle joint ligament injury during the game. After consuming the supplement for two weeks the subject fully recovered and returned regular sport routine.

Case 3: A 48 years old male diagnosed with ulcers (H. pylori infection) and prescribed a combination of antibiotics. In order to avoid consumption of antibiotics, the subject switched to the pharmaceutical composition (shilajit and Manuka honey) in moderation with mild diet. Dosage has been defined according to subject's weight: (0.25 g per kg of the body weight)/14 days) around three times a day, 30 min prior to a meal. After 2 weeks of continuous consumption of the supplement the subject felt significant health improvements. After following endoscopy the infection has not been detected.

Case 4: A 57 years old woman fractured her forearm as a consequence of accidental fall. After taking the supplement for 21 days, 3 times a day before the meals. First 7 days the subject doubled a dosage of the supplement, followed the remaining week with regular dosage. The subject reported significant improvements the next few days such as decreasing of puffiness and increasing mobility followed by general regeneration of the tissues.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments are chosen and described in order to best explain the principles of the present invention and its practical application, and to thereby enable others skilled in the art to best utilize the present invention. It is understood that various omissions and substitutions of equivalents are contemplated as circumstances may suggest or render expedient, but such omissions and substitutions are intended to cover the application or implementation without departing from the scope of the claims of the present invention.

What is claimed is:

1. A method to extract a pharmaceutical composition from a therapeutic compound consisting of selenium and minerals, humic substances, and dibenzo-alpha-pyrones, the method comprising:
adding distilled water to the therapeutic compound if residual moisture content of the therapeutic compound is found less than 40%;
heating and mixing the therapeutic compound to a temperature ranging between 25° C. to 37° C. using a heating source to form a homogenous composition;
adding and mixing a predefined amount of at least one plant extract to the homogenous composition;
cooling down the homogenous composition with the added at least one plant extract at room temperature of about 70° F.;
placing the homogenous composition with the added at least one plant extract into a dehydration system to reduce the residual moisture content of the homogenous composition from 40% or more to 17%-25%;
remixing the homogenous composition after 12-18 hours to enhance the evenness of texture and prevent precipitation of deposit in the homogenous composition; and
storing the homogenous composition at a temperature ranging between 10-24° C. (50-75° F.) to obtain the pharmaceutical composition.

2. The method of claim 1, wherein the therapeutic compound is a purified shilajit.

3. The method of claim 2, wherein the purification of the therapeutic compound is done using at least following steps:
crushing raw form of the therapeutic compound obtained from rocks in high altitudes of mountains into smaller particles;
dissolving the smaller particles of the therapeutic compound in water; and
applying multi-stage filtration on the dissolved smaller particles of the therapeutic compound to obtain purified of the therapeutic compounds for further processing.

4. The method of claim 1, wherein the heating source is at least a non-electrical heating source, or an electric heating source.

5. The method of claim 4, wherein the non-electrical heating source is a water bath.

6. The method of claim 1, wherein the at least one plant extract is a natural sweetener.

7. The method of claim 1, wherein the at least one plant extract is a Manuka honey.

8. The method of claim 1, wherein the at least one plant extract is a Black seed honey.

9. The method of claim 1, wherein the at least one plant extract is a combination of a Manuka honey, and a Black seed honey added in equal proportions.

10. The method of claim 1, wherein the therapeutic compound and the at least one plant extract includes similar or dissimilar viscosity at the room temperature based on the residual moisture content in the therapeutic compound.

11. The method of claim 1, wherein the homogenous composition is placed into the dehydration system for about 12-24 hours for reducing the residual moisture content of the homogenous composition from 40% or more to 17%-25%.

12. The method of claim 1, wherein the homogenous composition with the added at least one plant extract is placed into the dehydration system maintaining a temperature lower or equal to 37° C.

13. The method of claim 1, wherein the pharmaceutical composition comprising about 65% of the at least one plant extract, and about 35% of the therapeutic compound.

14. The method of claim 1, wherein the dehydration system is selected from a group consisting of a vacuum assisted dehydration system, a sunlight assisted dehydration system, and a conventional dehydration system.

15. A pharmaceutical composition comprising a therapeutic compound having selenium and minerals, humic substances, and dibenzo-alpha-pyrones,
characterized in that, the therapeutic compound is mixed with at least one plant extract having polyphenolic composition and other bioactive compounds, such as glyoxal and methylglyoxal, to obtain the pharmaceutical composition for a medical treatment; and wherein the pharmaceutical composition so obtained comprising about 65% of the at least one plant extract, and about 35% of the purified therapeutic compound.

16. The pharmaceutical composition of claim 15, wherein the therapeutic compound comprises of a purified shilajit.

17. The pharmaceutical composition of claim 15, wherein the at least one plant extract is a natural sweetener.

18. The pharmaceutical composition of claim 15, wherein the at least one plant extract is selected from a group consisting of a Manuka honey, a Black seed honey, and a combination of the Manuka honey and the Black seed honey added in equal proportions.
